(19) United States
(12) Patent Application Publication
(10) Pub. No.: US 2001/0053371 A1
(43) Pub. Date: Dec. 20, 2001

(54) METHOD FOR DIAGNOSING, IMAGING, AND TREATING TUMORS USING RESTRICTIVE RECEPTOR FOR INTERLEUKIN 13

(76) Inventors: WALDEMAR DEBINSKI, HERSHEY, PA (US); JAMES R. CONNOR, HERSHEY, PA (US)

Correspondence Address: J RODMAN STEELE JR, QUARLES AND BRADY, 222 LAKEVIEW AVENUE SUITE 400, P O BOX 3188, WEST PALM BEACH, FL, 334023188

(21) Appl. No.: 09/226,794

(22) Filed: Jan. 7, 1999

(51) Int. Cl. A61K 39/00; A61K 39/395; A61K 38/00; C07K 2/00; C07K 17/00

(52) U.S. Cl. 424/277.1; 424/85.4; 424/183.1; 530/300; 530/350

(57) ABSTRACT

Disclosed is a method of inhibiting the growth of tumors bearing IL13-specific receptors. Included among this class of tumors is glioblastoma multiform (GBM), a rapidly progressing brain tumor for which there is currently no effective treatment available. In the disclosed method, a chimeric cytotoxin comprising an IL13 receptor-binding moiety and a cytotoxic moiety is delivered into a mammalian subject having a tumor bearing IL13-specific receptors. All studied human GBM specimens abundantly express the IL13-specific tumor.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

The identification of tumor-specific cellular markers has proven extremely valuable in the diagnosis and treatment of certain types of malignancy. Cellular markers that occur on the plasma membrane or in a membrane receptor are particularly useful. Antibodies specific for tumor cell markers or ligands that bind specifically to a tumor cell receptor have been successfully used in diagnostics, including both the characterization of excised tissue samples and in vivo imaging. Tumor-specific antibodies and ligands have also been used in the targeted delivery of cytotoxic molecules to specific tumor cells.

Glioblastoma multiforme (GBM) is a rapidly progressing brain tumor for which there is no effective treatment available (1). Glioblastoma multiforme tumors are characterized by striking heterogeneity. Because of this heterogeneity, it has proven very difficult to identify suitable GBM markers that are essentially ubiquitous among and specific for GBM tumors for use in diagnostics and the development of targeted GBM-specific pharmaceuticals.

Efforts to identify a GBM brain tumor-specific plasma membrane antigen or receptor that is expressed by a majority of these tumors have been unsuccessful. Because of the therapeutic and diagnostic potential of tumor-specific antigens and receptors, there has been continuous and thus far, unsuccessful, research directed toward identifying an antigen, or a receptor for a growth factor/cytokine, that is present in more than 50% of high grade gliomas and not found in normal tissues to any significant degree. Due to the morphological heterogeneity of GBM tumors, it actually seemed unlikely to identify such a potential target receptor/antigen.

An epidermal growth factor receptor (EGFR) mutant, designated EGFRvIII, was identified as a potentially promising marker. However, it is expressed by only about 40% of malignant gliomas, and it is found to occur in solid tumors other than GBM (2). Furthermore, it was discovered that expression of EGFRVIII is lost by all cancer cells in culture, and it is not known if the process of receptor loss/gain takes place within tumors in vivo (2). The non-mutated EGFR is present on a subset of malignant human gliomas as well (˜40%), although it becomes less prevalent with the progression to GBM. In contrast, many normal cells express the EGFR in high numbers (2).

GBM tumors have been found to express a ubiquitous physiological transferrin receptor (TfR). Although TfR lacks specificity and therefore is unsuitable for use in diagnostics, TfR has been shown to be clinically tractable using anti-cancer cytotoxins (3).

A chloride channel has been found in a vast majority of tested human gliomas but not in normal tissues (4). The role of this channel in the pathogenesis has not been elucidated, nor has its potential utility in the diagnosis and treatment of GBM been evaluated.

There are currently no known GBM markers suitable for use in diagnosis and imaging, and which would also serve as a GBM-specific target for therapeutic deliveries. What is needed in the art is a tumor-specific marker that is found on a majority of GBM tumors.

### BRIEF SUMMARY OF THE INVENTION

One aspect of the present invention is a method of inhibiting the growth of a tumor in a mammalian subject, the tumor having an IL13-specific receptor, comprising the step of delivering into the subject a molecule comprising an IL13 receptor-binding moiety and a cytotoxin moiety in an amount effective to inhibit tumor growth.

Another aspect of the present invention is a method of imaging a tumor in a mammalian subject, the tumor having an IL13-specific receptor, comprising the steps of: delivering into the subject labeled IL13 receptor-binding molecules in an amount effective to image tissue; and scanning the subject to determine the distribution of the labeled IL13 receptor-binding molecules.

The present invention is also a method of evaluating an excised mammalian tissue sample for the presence of tumor tissue bearing an IL13-specific receptor comprising the steps of: exposing the tissue to an amount of a detectably labeled IL13 receptor-binding molecule moiety effective to bind to IL13-specific tumor tissue; and

examining the sample for the presence or absence of labeled IL13.

It is an object of the present invention to provide a method of inhibiting the growth of tumors bearing IL13-specific receptors.

It is a further object of the present invention to provide a method of in vivo detection of a tumor having an IL13-specific receptor in a mammalian subject.

Another object of the present invention is to provide a method of identifying tumor tissue bearing an IL13-specific receptor in excised mammalian tissue.

It is a feature of the present invention that a cytotoxic molecule may be specifically targeted to a tumor cell bearing an IL13-specific receptor.

Other objects, features, and advantages of the present invention will become apparent from the specification and claims.

### BRIEF DESCRIPTION OF THE SEVERAL VIEWS OF THE DRAWINGS

FIG. 1A shows survival of GBM explant cells (G3) treated with hIL13 cytotoxin (hIL13-CTX) alone (shaded circles), or in the presence of hIL13 (open circles) or hIL4 (triangles).

FIG. 1B shows survival of HUVEC treated hIL13-CTX (circles) or a cytotoxin that targets TfR (Tf CTX) (squares).

FIG. 2 shows the effect of intratumoral injection of hIL13-CTX on subcutaneous U373 MG tumor volume as a function of time. Arrows indicate time of injection.

FIG. 3 shows the effect of intratumoral injection of hIL13-CTX on subcutaneous U251 MG tumor volume as a function of time. Arrows indicate time of injection.

FIG. 4 shows fraction survival over time of SCID mice bearing U251MG glioma tumors injected with IL13-PE4E (open circles) or saline (shaded circles).

### DETAILED DESCRIPTION OF THE INVENTION

Work in our laboratory has established the presence of large numbers of a receptor specific for interleukin 13 (IL13) on established human malignant glioma cell lines and on freshly explanted cells cultured from a resected GBM tumor (5,6). Permanently cultured malignant glioma cells were found to have up to 30,000 IL13 binding sites per cell, whereas freshly explanted GBM cells may have as many as 500,000 binding sites per cell (5,6). The IL13-specific receptor is also expressed by certain other tumor cells (U.S. Pat. No. 5,614,191). The IL13-specific receptor is an attractive candidate for targeting malignant cells using a modified IL13 ligand to facilitate in vivo diagnosis and treatment of glioblastoma multiforme, as well as other tumors that express the IL13-specific receptor in vivo.

The present invention relates generally to methods of identifying tumors bearing a more restrictive IL13-specific receptor and to methods of inhibiting the growth of tumors bearing an IL13-specific receptor.

Accordingly, one aspect of the present invention is a method of inhibiting the growth of a tumor in a mammalian subject, the tumor having an IL13-specific receptor. The method comprises the step of delivering into the subject an amount of a molecule effective to inhibit tumor growth, the molecule comprising an IL13 receptor-binding moiety and a cytotoxin moiety.

Another aspect of the present invention is a method of imaging a tumor in a mammalian subject, the tumor having an IL13-specific receptor, comprising the steps of: delivering into the subject an amount of a detectably-labeled IL13 receptor-binding molecules effective to image tissue; and scanning the subject to determine the distribution of the labeled IL13 receptor-binding molecules.

The present invention includes a method of identifying the presence of tumor tissue bearing an IL13-specific receptor in an excised mammalian tissue sample comprising the steps of: exposing the tissue to an amount of a detectably-labeled IL13 receptor-binding molecule effective to bind to IL13-specific tumor tissue; and examining the sample for the presence or absence of bound, labeled IL13 receptor-binding molecules.

Another aspect of the present invention is a nucleotide fragment comprising a coding sequence for an IL13-specific receptor. Identification and characterization of this fragment will allow determination of at least one genetic locus implicated in GBM tumor proliferation. Assignment of the receptor gene to a specific locus will facilitate the identification of other associated sequences that may play a role in the pathogenesis of this disease.

IL13 is a regulatory cytokine that exhibits homology to IL4. Like IL4, IL13 has anti-inflammatory properties (7). Both hIL13 and hIL4 exert their effects by binding to a functional IL13/IL4 receptor that is present on selected normal tissues, and which is over-expressed on some adenocarcinomas (8,9). Surprisingly, hIL4 neither neutralizes the action of IL13 cytotoxins nor competitively inhibits in vitro binding of IL13 to any of the tested malignant glioma cells (5,6). Based on these findings, we hypothesized the presence of a more restrictive IL13-specific receptor on malignant glioma cells.

An “IL13-specific receptor” as used herein is a receptor that binds to IL13 to a much greater extent than it binds IL4. Preferably, the affinity of the IL13-specific receptor for IL13 is at least 1000× higher than its affinity for IL4.

By an “IL13-specific receptor-binding molecule or moiety” it is meant any molecule or molecular moiety that binds to an IL13-specific receptor with greater affinity than IL4 binds the receptor, or a molecule or molecular moiety that binds to an IL13-specific receptor with greater affinity than it binds other proteins including the functional IL13/4 receptor. For example, an IL13-specific molecule or moiety could include an IL13 molecule, or portion thereof, or a mutagenized IL13 molecule, or portion thereof, or an antibody specific for an IL13-specific receptor.

In vitro studies have demonstrated that cultured malignant glioma cells are extremely sensitive to cytotoxic proteins comprising hIL13 and a cytotoxin, including derivatives of a bacterial toxin, such as Pseudomonas exotoxin (PE) PE38QQR or PE4E (5,6,8) or engineered Diphtheria toxin (W. Debinski, unpublished material).

The results of experiments using cultured malignant gliomas suggested to us that hIL13R is a promising candidate for the diagnosis, imaging, and therapeutic targeting of malignant tumors bearing IL13-specific receptors, including malignant gliomas. However, the potential importance of a cancer-associated receptor or antigen depends exclusively on its tumor representation versus expression in normal tissue in situ. It is noteworthy that recent studies on GBM showed that an antigen of high specificity that is present clinically is completely lost in cell culture (20) or, in a reverse scenario, over-expression of a molecule seen in vitro does not correspond to an in situ situation (11). Therefore, in order to evaluate the possible clinical importance of the IL13-specific receptor as a candidate marker or target in the diagnosis or treatment of GBM, it was essential to demonstrate that hIL13 binding sites are present in GBM but not in normal brain tissues using freshly-preserved surgical specimens. The potential importance of these receptors was further evaluated by conducting preclinical tests using cytotoxins linked to an IL13-specific receptor-binding moiety.

The examples below demonstrate that a labeled IL13 receptor-binding molecule can be used to visualize IL13-specific receptors on GBM tumors in freshly excised tissue, because GBM tumors bind IL13 to a much greater extent than does normal tissue.

As detailed in the examples, tissue samples were evaluated for binding of IL13 in situ and used to establish GBM cell cultures. GBM tumor cells were found to bind ¹²⁵I-hIL13 extensively, relative to binding by normal brain cells. Cultured GBM cells probed with ¹²⁵I-hIL13 and subjected to autoradiography were shown to bind ¹²⁵I-hIL13 extensively, whereas cultured normal human umbilical vein endothelial cells (HUVEC) did not.

We have previously demonstrated that hIL13-based cytotoxins kill potently established cultures of malignant glioma cells (5,6). To determine whether similar results could be obtained in vivo, the hIL13-based cytotoxins were constructed and tested for the ability to inhibit tumor growth in nu/nu athymic mice subcutaneously established xenographic GBM tumors from humans or scid mice bearing intracranial xenographic GBM tumors. As shown in the examples, the in vivo mice studies indicate that the hIL13-based cytotoxins were effective in inhibiting the growth of tumors bearing hIL13-specific receptors in vivo.

Modified IL13-specific Receptor Molecule for In Vivo Imaging and Chimeric Cytotoxin

We have discovered that IL13 binds to the GBM tumor cells with specificity. This feature allows targeting of IL13 to specific tumor cells bearing the IL13-specific receptor. An IL13 molecule can be modified to include a label or a cytotoxic moiety.

It is expected that any IL13 molecule, regardless of its source, may be used in the present invention because IL13 is conserved among species. It is further expected that an IL13-specific receptor-binding molecule could include an antibody specific for IL13-specific receptors. The present invention is intended to encompass a molecule having an IL13 receptor-binding moiety with substitutions, additions, and deletions, provided that such changes do not impair the ability of IL13 to bind to the IL13-specific receptor. It is anticipated that an IL13 molecule that is truncated from either the N-terminal region or the C-terminal region can be employed in the present invention, provided that the altered IL13 ligand retains the ability to bind to the IL13-specific receptor. It is well within the ability of one skilled in the art to create derivatives of IL13 using a cloned IL13 gene and standard molecular biology techniques. These IL13 derivatives could be detectably labeled and evaluated for the ability to bind to IL13-specific receptors using the teachings disclosed herein. It is envisioned that one wishing to obtain an IL13 molecule for use in the present invention could do so by synthesizing the portion of the gene that specifies binding to an IL13-specific receptor, expressing the gene, and purifying the expression product.

To detect the presence of IL13-specific receptor-binding molecules binding to an IL13-specific receptor in a freshly excised tissue, the IL13-specific receptor-binding molecule may be detectably labeled with any conveniently detectable label, including radioisotopes, fluorophores, chromophores, or enzymes such as horseradish peroxidase. In the examples, IL13 was labeled with ¹²⁵I. It is expected that an IL13-specific receptor-binding molecule labeled with any radiolabel, fluorophore, chromophore, or enzyme with readily detectable activity could be successfully employed in the practice of the present invention.

For in vivo imaging of tumors bearing IL13-specific receptors, an IL13-specific receptor-binding molecule can be labeled with a scannable radiolabel, such as alpha electron emitters (e.g., bismuth), beta electron emitters (e.g., rhenium, iodine 131), or Auger electron emitters (iodine 125), delivered into the subject, and the subject can then be scanned.

To obtain an IL13 receptor-binding molecule having a cytotoxic moiety for use in targeted chemotherapy, a cytoxic moiety may be joined to a full length or truncated IL13-specific receptor-binding molecule using standard chemical or molecular biological techniques. Suitable cytotoxic moieties, which are discussed below, can include any cytotoxic moiety that is susceptible to being joined to an IL13 receptor-binding molecule and which retains cytotoxic activity when attached to IL13. Any method of joining the IL13 receptor-binding and cytotoxic moieties can be used. For example, the moieties may be conjugated by chemical means, of which numerous methods are known to the art. When the cytotoxic moiety is a cytotoxic peptide, the toxin can most conveniently be joined to the IL13 receptor-binding moiety using known molecular biological means.

Cytotoxic moiety

One skilled in the art would appreciate that the present invention could be practiced using any number of cytotoxins joined to the IL13 receptor-binding moiety. Numerous cytotoxic moieties and methods of conjugating these molecules to proteins are known to the art. For example, cytotoxic radionuclides, ribosome inhibitors, methotrexate, plant toxins, and bacterial toxins have been used to create immunotoxins. In the examples below, the chimeric cytoxic molecules employed in the in vivo assay included the bacterial toxin Pseudomonas exotoxin (PE) PE4E or PE38QQR as the cytotoxic moiety. A genetically engineered Diptheria toxin was found to inhibit the growth of cultured GBM cells, and it is expected that this toxin would be effective in vivo as well. It is expected that any plant, bacterial, or animal toxin effective in inhibiting cell growth can be used in the present invention.

Preferred chimeric IL13 cytotoxin construct

In the examples below, the IL13 receptor-binding moiety is the full length human IL13 molecule, fused to a cytotoxic peptide. Preferably, the cytotoxic peptide is selected from the group consisting of an engineered Diptheria toxin or a Pseudomonas exotoxin, most preferably PE4E or PE38QQR.

IL13-specific Receptors in Other Tumor Cells

It is expected that the method of the present invention may be effective in inhibiting the growth of any tumor bearing large numbers of IL13-specific receptors. For example, this method may be effective in inhibiting the growth of human renal cell carcinomas and AIDS-associated Kaposi's sarcomas, which have been found to bear IL13-specific receptors in vitro. Using the teaching disclosed herein, one skilled in the art could easily test the in vivo efficacy of this method using a suitable animal model having any xenograft tumor bearing IL13-specific receptors.

Protocol for Administering the IL13-based Cytotoxin

Athymic mice bearing subcutaneously established xenograft tumors or SCID mice bearing xenograft intracranial tumors were used in in vivo assays to test the ability of a cytotoxin targeted for the IL13 receptor to inhibit growth of tumors bearing hIL13-specific receptors. This is a mammalian model system that has been found to be useful in preclinical trials to evaluate the in vivo efficacy of chemotherapeutic agents. Therefore, it is reasonable to expect that a cytotoxin directed toward the hIL13 receptor would be effective in inhibiting the growth of tumors bearing hIL13-specific receptors in other mammals, including humans.

In the examples below, the IL13-cytotoxin chimeric proteins were delivered to the tumor via intratumoral injection, because intratumoral delivery has been shown to offer certain advantages over other delivery means in the treatment of central nervous system (CNS) malignancies (3,12). Intratumoral (IT) injection overcomes the problems associated with delivering pharmaceuticals across the blood-brain barrier. It is expected that intracranial injection could also be used to deliver the chimeric cytotoxins for treatment of CNS malignancies. Other modes of administration, including for example intravenous (IV) or intramuscular (IM) injection, or oral administration, would be expected to be effective in delivering the chimeric cytotoxins to tumors located at sites outside the CNS.

In the Examples below, treatment of mice bearing subcutaneous human glioma tumors with five or six intratumoral injections of from 0.1 to 0.5 ug administered at 48 hour intervals was effective in reducing tumor volume in a dose dependent manner. The tumors in mice that received 0.5 ug injections of cytotoxin were reduced in size relative to the initial tumor volume. In contrast, the tumors in mice treated with the vehicle alone continued to grow over time to about two to four times the original volume. Mice that received intermediate levels of cytotoxin (0.1 ug) demonstrated a reduction in the growth of the tumors, with a tumor volume of only about 50% of that of the mice treated with the vehicle.

An effective amount of cytotoxin is that amount which is sufficient to exhibit a cytostatic or cytotoxic effect. A cytostatic effect is evidenced by a reduction in the rate of growth of the tumor relative to a comparable untreated tumor. Arresting the progression of tumor growth will likely afford a patient suffering from GBM some benefit. Preferably, administration of the cytotoxin will reduce the rate of tumor growth by at least 25%. More preferably, administration of the cytotoxin will reduce the rate of tumor growth by at least 50% or even as much as 100%.

A cytotoxic effect is manifested as a reduction in tumor volume. Administration of cytotoxin may not only reduce the rate of tumor growth, but may actually cause a reduction in tumor size, or even eliminate the tumor mass. Although eliminating the tumor mass altogether would be preferable, it should be appreciated that even slowing the rate of growth of this rapidly progressing tumor may benefit the patient. Preferably, the tumor volume is reduced by at least 10%. More preferably, the tumor volume is reduced by 25%, or even as much as 50%. Still more preferably, the tumor mass is reduced by up to 100%.

Treatment of mice bearing an intracranial glioma with two intratumoral injections of 0.2 ug at a one week interval was effective in reducing wasting and extending longevity of the mice. It should be appreciated that one could vary the amount of cytotoxin administered as well as the number and spacing of the treatments and achieve effective reduction in tumor volume. Delivery can be done by prolonged infusion over the time using delivery pumps capable of infusing the dosage over a period of time from one day to one week either intratumorally or intravenously. Optimization of dosages and dosage schedules is well within the ability of one skilled in the art. It is expected that suitable dosages will depend on the means of delivery. For intratumoral injections, a dosage of from about 0.001 mg to about 1.0 mg is expected to be appropriate for humans, depending upon the size of the tumor when treatment is initiated.

Pharmaceutical Compositions

In the examples below, the IL13-based cytotoxin was delivered in a small volume of PBS containing 0.1% BSA. Any suitable pharmaceutical carrier can be employed in the present invention. The formulation chosen will depend on the mode of administration. For example, if oral administration is indicated by the location of the tumor, the IL13-based cytotoxin may be encapsulated in liposomes. Normal saline may be used as a carrier for IM, IV, or IT injection of the IL13-based cytotoxin, alone or together with BSA or preferably HSA.

The following nonlimiting examples are intended to be purely illustrative.

### EXAMPLES

Preparation of ¹²⁵I-labeled hIL13

Recombinant hIL13(8) was labeled with ¹²⁵I by using IODO-GEN reagent (Pierce) according to the manufacturer's instructions. The specific activity of ¹²⁵I-hIL13 ranged from 40 to 852 μCi/g of protein. Six different batches of labeled hIL13 were used in this study.

Sample Collection and Preparation

Normal human brain tissues were obtained either from lobectomies and snap-frozen for analysis or post-mortem from the Harvard Brain Tissue Research Center. Glioblastoma multiforme tumor samples were obtained from the operating rooms at Hershey and Birmingham. Samples included tissue from various areas of the normal brain, including the motor cortex, white matter, hippocampus, sub-ventricular white matter, and temporal lobe. Among the twenty-three patients evaluated, there were 12 females and 11 males, varying in age from 16 to 79 years. The GBM obtained from 3-month and 1year old children (GBM #10 and GBM #22, respectively) were not included in this study. All studies involving human specimens were approved by the respective Human Subjects Protection Offices at the Penn State College of Medicine (Protocol No. IRB 96-123EP) and University of Alabama Medical School.

The GBMs were processed randomly from among the samples preserved at UAB or sequentially from among the samples obtained at Hershey. Serial tissue sections (10 μm) were made using a cryostat, thaw-mounted on chrom-alum coated slides, and stored at 4° C. until analyzed (13).

Establishment of Glioblastoma Multiforme Cell Cultures

Pathology-proven surgical specimens of glioblastoma multiforme were collected and transferred to our laboratory under sterile conditions. Peripheral and necrotic tissues were excised and the remaining tissue minced using a scalpel. Tumor tissue was incubated in a cocktail composed of collagenase type II and IV, DNAase I, and NuSerum/DMEM, at 37° C. with constant shaking for 45 min. Cells were layered onto Ficoll-Paque, centrifuged for 35 min at 400× g and 1820° C. The cells were resuspended in 3× volume of balanced salt solution and centrifuged (100× g, 18-20° C., 10 min). The cell pellet was washed again, resuspended in RPMI 1640/25 mM HEPES with L-glutamine supplemented with 10% FBS, 0.1 ng/ml L-cystine, 0.02 mg/ml L-proline, 0.1 mg/ml sodium pyruvate, HT supplement, and antibiotics. The cells were transferred to 100-mm plates and incubated at 37° C. in 95% O₂/5% CO₂ humidified atmosphere.

Once in culture, early passages of the GBM cells were used for autoradiography concomitantly with normal human umbilical vein endothelial cells (HUVEC), or treated with an hIL13-based cytotoxin.

Bacterial transformation

E. coli BL21 (XDE3) cells were transformed with plasmids of interest and cultured in Terrific Broth (DIFCO Laboratories, Detroit, Mich.). Procedures for recombinant protein isolation and purification has been previously described (5,6,8).

Binding Distribution of ¹²⁵I-labeled hIL13 to brain tissue

Adjacent serial sections were pre-incubated for 30 min at 22° C. in binding buffer (200 mM sucrose, 50 mM HEPES, 1% BSA, 10 mM EDTA) alone, or in binding containing a 100- to 500-fold molar excess of unlabeled hIL13 or hIL14, or transferrin. Following preincubation, sections were incubated for one hour at 22° C. with 1.0 nM ¹²⁵I-hIL13. Nonspecifically bound radioligand was removed by rinsing sections in four consecutive changes (5 minutes each) of ice-cold 0.1 M PBS. At least two sections of each of the tissue specimens were assayed for ¹²⁵I-hIL13 binding specificity. After drying, labeled sections were apposed to Kodak autoradiography film at −65° C. for 8 hr to 11 days.

Some autoradiographic sections were coated with autoradiography type NTB3 emulsion (Eastman Kodak Co., New Haven, Conn.) and exposed for three to four days in sealed light-tight boxes at 4° C. The preparation was then developed for 5 minutes with D19 Kodak, rinsing in distilled water for 2 minutes, fixed in Kodak fixer for 4 minutes, and washed in distilled water for 2 minutes. Subsequently, the sections were stained with H&E and analyzed under light microscope (×10 or ×20 magnification) for the presence of silver grains or using epifluorescence optics.

For autoradiography on cultured cells, the cells (approximately 1×10⁴) were placed on a sterile glass slide in a small volume of media and maintained for three days at 37° C. to allow attachment. The slides were washed in two changes of 0.1 M PBS, fixed with ethanol, rinsed again with 0.1 M PBS, and processed for autoradiography, as described above.

Autoradiographic images were scanned using HP ScanJet 4C flat bed scanner (Hewlett-Packard, Boise, Id.) at 200 dpi. Sections were analyzed and mounted using the Paint Shop Pro 5 program (Jasc Software, Minnetonka, Minn.).

Scanning of the autoradiographic images revealed that twenty-two out of twenty-three adolescent/adult GBMs studied bound ¹²⁵I-hIL13. The GBM tissues generally labeled densely and homogeneously for the ¹²⁵I-hIL13 binding sites. Preincubation of these samples with an excess of unlabeled hIL13 reduced binding of ¹²⁵I-hIL13, whereas an preincubation of the samples with an excess of recombinant hIL4 did not reduce signal intensity from ¹²⁵I-hIL13 binding. This finding indicates that hIL13 binds to a receptor that is unable to bind hIL4. These results are consistent with earlier results of in vitro studies that suggested the presence of an hIL4-independent GBM-associated hIL13R (5,6) in eight out of nine tested established malignant glioma cells and provide further evidence that the functional hIL13/4R of normal tissue is different from GBM-associated hIL13R.

Whereas most of the GBM specimen samples bound ¹²⁵I-hIL13 densely and homogeneously, it was found that binding of ¹²⁵I-hIL13 to GBM sample #6 was competed for by unlabeled hIL13 over a limited area of the section. Only the GBM #20 did not show any specific uptake of the isotope (W. Debinski, unpublished material). IL13 binding to GBM #15 was low relative to binding by the other GBM samples; however, an excess of hIL4 did not reduce binding by hIL13, indicating that the receptors of this sample are hIL13-specific (W. Debinski, unpublished material). In another test of specificity of the hIL13 binding to GBM, we tested the ability of Tf to compete with the binding of radiolabeled interleukin. We did not observe competition between Tf and hIL13 for binding sites in the five GBMs examined. In another set of experiments and test of specificity, GBM did not exhibit any measurable over-expression of the receptor for hIL4. Thus, autoradiographic analysis revealed that a large percentage GBM tumors express detectable amounts of an IL13-specific receptor.

In order to visualize the areas of GBM sections that bind the labeled hIL13, we examined the autoradiograms by light microscopy and with epifluorescence optics. The autoradiograms showed that ¹²⁵I-hIL13 specific binding was distributed relatively uniformly over the whole area of GBM specimens. Light-microscopic analysis revealed that the vast majority of tumor cells was stained with silver grains. This strongly supports the notion that majority of GBM cells possess this more restrictive IL13R in situ. Because autoradiography suggested that one of the GBM tumors did not appear to bind ¹²⁵I-hIL13 and a few GBM tumors exhibited more heterogenous binding, H&E stained sections corresponding to the autoradiographic images were examined. Those GBM samples that did not show specific binding of ¹²⁵I-hIL13 or which demonstrated heterogeneous binding were completely or partially acellular or necrotic, whereas the GBM specimens that bound ¹²⁵I-hIL13 avidly had the cellular organization preserved. Thus, it is plausible that all GBMs over-express hIL13R, but detection of the receptor is reduced in acellular or necrotic samples. In preliminary studies, other types of brain tumors, including lower grade gliomas, meningiomas, and medulloblastomas, did not demonstrate this pattern of hIL13 binding to GBM (Debinski, unpublished).

GBM explant cells bound ¹²⁵I-labeled hIL13, but not hIL4, which indicates that the IL13-specific receptors are not lost in cultured cells. In contrast, HUVEC did not bind ¹²⁵I-labeled hIL13.

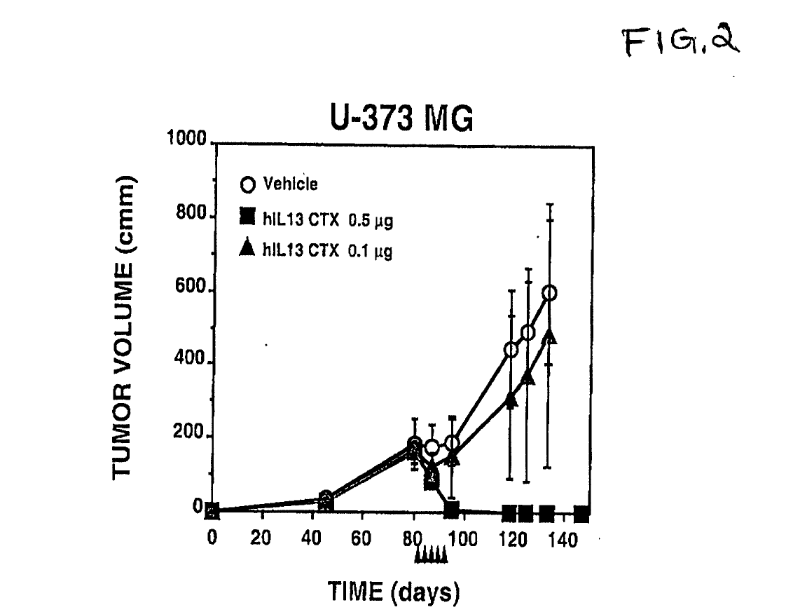

Samples of normal human brain tissue did not show appreciable affinity for ¹²⁵I-hIL13. All six examined specimens showed the same low retention of the ¹²⁵I-hIL13 relative to the labeling of GBM tumors, and this low level binding was changed only marginally in the presence of an excess of either cold hIL13 or hIL4 (FIG. 2). These results provide further evidence that the IL4-independent hIL13R detected on GBM is a tumor-specific marker. The expression of an IL4-independent hIL13R by GBM cells has been shown by us to be of significance for further translation of this finding into clinical applications (14). In summary, the GBM-associated hIL13R represents uniquely new marker for diagnostic labeling of cells and potentially for imaging, and a target for delivery of cytotoxic or cytostatic therapies to this most devastating malignancy. Our study supports the idea that a malignancy as heterogeneous as the GBM could be characterized by the expression of specific molecules indeed (4, 15, 16). Further investigations based on the knowledge of those molecules should help also in deciphering the pathogenesis of GBM. (W. Debinski, unpublished material).

Assay of Chimeric Toxin Cytotoxicity in Cultured Cells

The cytotoxic activity of chimeric toxins was tested as follows. The GBM cells (sample #3)(5×10³ cells per well) were plated in a 96-well tissue culture plate in 150 μL of media. Various concentrations of hIL13-PE38QQR and a Tf cytotoxin (HB21xF(ab′)-PE38QQR)(11) were prepared in PBS/0.1% BSA and 25 μl of each dilution was added to cells 18-24 hr after cell plating. The cells were incubated for 48 hr at 37° C. and the cytotoxicity was determined using a colorimetric MTS [3-(4,5-dimethylthiazol-2-yl)-5(3-carboxymethoxyphenyl)-2-(4-sulfophenyl)-2H-tetrazolium, inner salt]/PMS(phenazine methasulfate] cell proliferation assay. MTS/PMS was added at half the final concentration as recommended by the manufacturer (Promega, Madison, Wis.). The cells were incubated (4 hr) and absorbency was measured at 490 nm for each well using a microplate reader (Cambridge Technology, Inc., Watertown, Mass.). Wells containing cells treated with cycloheximide (10 mM) or wells having no viable cells remaining served as a background for the assay. For blocking studies, recombinant interleukins or their mutants were added to cells for 60 min before the addition of cytotoxins. Data were obtained from the average of quadruplicates and assays were repeated several times.

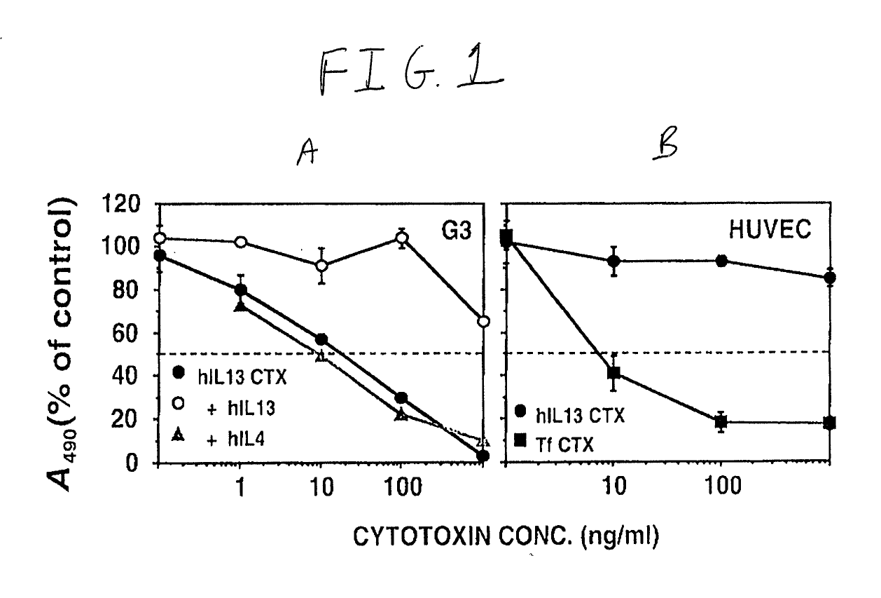

As shown in FIG. 1A, GBM explant cells are very sensitive to a hIL13 cytotoxin in a dose-dependent fashion. This cytotoxic effect is hIL13R-specific, as evidenced by neutralization by an excess of hIL13, but not of hIL4 (FIG. 1A). Again, the lack of interaction with hIL4 appears to be a hallmark of GBM-associated hIL13R as it was observed for the first GBM explant cells examined (6) and also for cells explanted from GBM specimen #5 (W. Debinski, unpublished material). Furthermore, the hIL13 cytotoxin did not affect HUVEC (FIG. 1B). This is due to a very low number of hIL13 binding sites on normal endothelial cells (10). A similar lack of susceptibility to a hIL13 cytotoxin was seen in freshly cultured mixed glial cells (W. Debinski, unpublished material). Not surprisingly, a cytotoxin that targets TfR (11) did kill HUVEC potently at an IC₅₀ of <10 ng/ml (FIG. 1B). This is in a range of killing potency of the anti-TfR cytotoxin observed for some glioma cells in vitro (W. Debinski, unpublished material). Moreover, normal endothelial cells contribute significantly to the strong autoradiographic picture of Tf binding sites within normal brain (18). The IL4-CTX also killed potently HUVEC cells (IC₅₀ of ˜25 ng/ml), which is consistent with IL4 having an affinity for hIL13/4R that is at least two orders of magnitude higher than the affinity of IL13 for hIL13/4R.

Effect of Chimeric Toxins on GBM Tumor Size in Mice

The human malignant glioma U-373 MG and U251-MG cells were implanted subcutaneously into 5 to 6-wk old female nu/nu athymic mice (6×10⁶ cells per mouse) on day 0. After large established tumors were formed, tumors and they were measured with a caliper, treatments including 4-5 mice per group were initiated. Tumor volume was calculated using the formula volume=length×^{width}×0.4 (14). The Institutional Animal Care Committee at the Penn State College of Medicine has approved the protocol.

Treatment with IL13-based Toxins Extends Life of SCID Mice Bearing Intracranial Tumors

Intracranial tumors were induced in CB-1.7 SCID mice by placing the mice on a stereotactic frame and injecting the mice intracranially with 1×10⁶ U-251 MG cells in a volume of 5 μl using Hamilton syringe, under anesthesia. At seven and fourteen days after tumors were induced, each mouse was re-operated and received an intratumoral injection of 0.2 μg of hIL13-PE4E or PBS in a 5 μl volume (10 mice per group). Mice that had become moribund or had lost more than 25% of body weight were euthanized. Median survivals were computed by Kaplan-Meier analysis. The Institutional Animal Care Committee at the University of Alabama at Birmingham has approved the protocol.

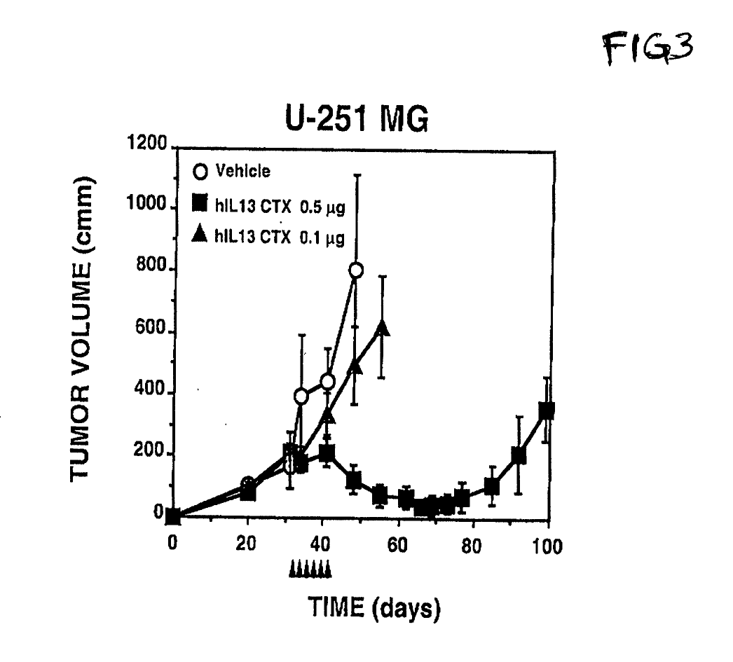

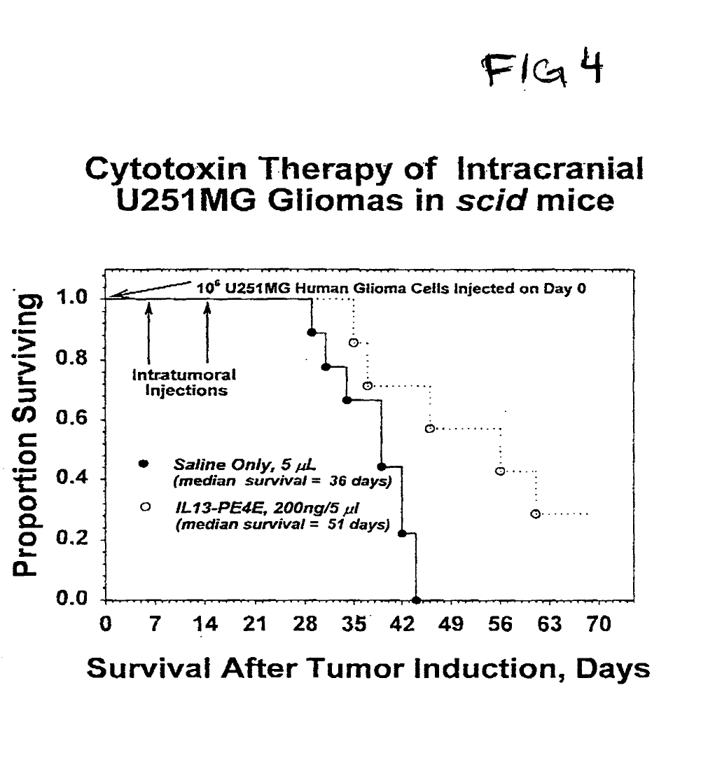

A cytotoxin that targets the hIL13R can produce dramatic anti-tumor effect in vivo. We used intratumoral injections of the cytotoxin, because intratumoral delivery has recently been shown to be a promising approach in the treatment of central nervous system malignancies and it offers several advantages over systemic delivery mechanisms (3,12). Because IL13 is not species-specific, the mouse model chosen in this study is more representative of a clinical situation. We treated nu/nu athymic mice bearing established subcutaneous (s.c) xenografts of two human malignant gliomas, U-373 MG (FIG. 2) and U-251 MG (FIG. 3), or scid mice with established intracranial (i.c.) xenografts of U-251 MG (FIG. 4), with either the vehicle or hIL13 cytotoxin. The treatment of U-373 MG s.c. tumors started on day 80 post tumor implantation, and on day 10 for U-251 MG tumors when the tumors were ˜200 cmm in size (˜8×8×8 mm). We had previously observed that tumors around 50 cmm can be cured with a hIL13 cytotoxin (W. Debinski, unpublished material). We found that 5 i.t. injections of 0.5 μg of the cytotoxin every other day produced complete regression of U-373 MG tumors in all of the cytotoxin-treated mice with no signs of toxicity and one mouse remained free of tumor in the 0.1 μg-treated group of mice (FIG. 2). In the U-251 MG tumor model, 6 i.t. injections of 0.5 μg of the cytotoxin every other day regressed tumors in all mice and two out of five animals treated initially were free of tumor on day 141 of the experiment (FIG. 3). of importance, two i.t. injections of 0.2 μg per mouse of hIL13 CTX in the intracranial model of human glioma (U251 MG) resulted in a high significant prolongation of the mice survival and 30% were long-term survivors (FIG. 4).

In vivo Imaging Using Labeled IL13

We anticipate that it will be possible to image tumors in vivo by using a modified IL13 ligand such that the IL13 is detectably labeled. One skilled in the art would appreciate that the method of the present invention could be practiced using a variety of detectable labels and scanning or imaging means. For example, the IL13 ligand could be labeled with ¹⁸F or ¹¹C using standard techniques, delivered into the subject by a suitable delivery means, and the localization of the labeled molecule determined by Positron Emission Tomography (PET). Single Photon Emission Computed Tomography (SPECT) can be used for tumoral localization of ligands labeled with labels detectable by SPECT (e.g., ²⁰¹Tl or ^{99m}Tc). Magnetic Resonance Spectroscopy (MRI) can be employed in the detection of suitably labeled ligands (e.g., ligands labeled with ³¹P or ¹H, for example). We anticipate that such imaging would be useful in determining appropriate treatment for brain tumors, and for following the progress of chemotherapy in the treatment of CNS malignancies.

Identification of a Nucleotide Fragment Encoding an IL13-specific Receptor

We are currently working to identify a polynucleotide fragment that encodes at least one IL13-specific receptor protein. IL13 receptor protein has been partially purified from a lysate of GBM tumor cells and renal cell carcinoma cells by affinity chromatography using a column to which IL13 has been covalently linked to the resin. The lysate is applied to the column and the retained proteins are eluted using a low pH lysine buffer. The fractions containing proteins exhibiting affinity for IL13 are subjected to SDS-PAGE. Those proteins having a molecular weight in the range of from about 50 to about 80 kDa will be removed from the gel and subjected to partial amino acid sequencing. The information obtained from amino acid sequencing will allow the design and synthesis of degenerative oligonucleotides useful in the identification of at least one nucleotide fragment encoding an IL13-specific receptor protein. These oligonucleotides will be labeled and used to screen cDNA libraries, or will serve as primers to amplify cDNA coding sequences from mRNA using RT-PCR.

Once a polynucleotide fragment encoding an IL13-specific receptor is identified, further characterization can be performed. For example, the fragment or a portion thereof could serve as a probe to identify the genetic locus of the full length gene. Neighboring DNA sequences or genes will also be examined.

A nucleotide fragment encoding an IL13-specific receptor may be cloned and used in in vitro assays to evaluate transand cis-acting factors involved in regulating expression of the gene.

Information obtained by sequencing a nucleotide fragment encoding an IL13-specific receptor may be very useful in molecular modeling to identify a small molecule (e.g., a peptide, nucleic acid, or other compound) that will bind to the receptor. Such a molecule would be useful for dianostics, imaging, and drug delivery.

Developing antibodies against the IL13-specific receptor is another approach to identifying a nucleotide coding sequence encoding the IL13-specific receptor. A protein that binds IL13, but not IL4, has been cloned (Caput, et al. J. Biol. Chem. 271:16921, 1996). We suspect that this protein may correspond to the IL13-specific receptor. We propose to produce a recombinant, extracellular portion of this receptor and develop monoclonal antibodies against the protein. These antibodies can be used to identify clones expressing the protein, or to evaluate any crossreativity that may exist between IL13 and these monoclonal antibodies in binding to GBM tumor cells.

Since GBM is a high grade glioma, which at least in some instances is believed that may arise from low grade gliomas, the IL13-specific receptor may also serve as an indicator of cancer progression.

All cited publications are incorporated by reference herein.

The present invention is not limited to the exemplified embodiments, but is intended to encompass all such modifications and variations as come within the scope of the following claims.

### REFERENCES

1. Kleihus et al., Glia 15:211 (1995); “Reports from the Front”, Science 267:1414 (1995).

2. Moscatello et al., Cancer Res. 55:5536 (1995); Wilkstrand et al., Cancer Res. 55:3140 (1995); Lorimer et al., Clin. Cancer Res. 1:859 (1995).

3. Recht et al., Cancer Res. 50:6696 (1990); Recht et al., J. Neurosurg. 72:941 (1990); Youle et al., Nature Med. 3:1362 (1997).

4. Ullrich N. and H. Sontheimer, Am. J. Physiol. 270: C1511 (1996); Ullrich et al., “Human Astrocytoma Cells Express a Unique Chloride Current”, NeuroReports 7:343-347 (1996).

5. Debinski et al., Clin. Cancer Res. 1(Advances in Brief):1253 (1995).

6. Debinski et al., J. Biol. Chem. 271:22428, 1996.

7. McKenzie et al., Proc. Natl. Acad. Sci. 90:3735 (1993); Minty et al., Nature 362:248 (1993).

8. Debinski et al., J. Biol. Chem. 270:16775 (1995).

9. Obiri et al., J. Immunol. 158:756 (1997); Murata et al., Biochem. Biophys. Res. Comm. 238:92 (1997).

10. Bochner et al., J. Immunol. 154:799 (1995); Sironi et al., Blood 84:1913 (1994); Schnyder et al., Blood 87:4286-4295 (1996).

11. Zellner et al., “Disparity in Expression of Protein Kinase C α in Human Glioma Versuse Glioma-Derived Primary Cell Lines: Therapeutic Implications”, Clin. Cancer. Res. 4:1797-1802 (1998).

12. Wersall et al., Cancer Immunol. Immunother. 44:157(1997).

13. Hulet et al., “Characterization and Distribution of Ferritin Receptors in the Mouse Brain”, J. Neurochem. In press, 1998.

14. Debinski et al., Nature Biotech. 16:449 (1998); Debinski et al., Int. J. Cancer 76:547 (1998).

15. Jaworski et al, “BEHAB (Brain Enriched Hyaluronan Binding) is Expressed in Surgical Samples of Glima and in Intracranial Grafts of Invasive Glioma Cell Lines”, Cancer Res. 56:2293-2298 (1996).

16. Murphy et al., “The Human Glioma Pathogenesis-Related Proteins Structurally Related to Plant Pathogenesis-Related Proteins and its Gene Expressed Specifically in Brain Tumors”, Gene 159:131-135 (1995).

17. Debinski, W. and I. Pastan, Cancer Res. 52:5379 (1992).

18. Jefferies et al., Nature 312:162 (1984); Connor, J. R., unpublished material.

We claim:

1. A method of reducing the rate of growth of tumor cells in vivo in a mammalian subject, the tumor cells comprising an IL13-specific receptor, comprising the step of delivering into the subject a molecule having an IL13-moiety and a cytotoxic moiety in an amount effective to reduce the rate of growth of tumor cells.

2. The method of claim 1, wherein the tumor cells are glioblastoma multiforme cells.

3. The method of claim 1, wherein the rate of tumor growth is reduced by at least 25%.

4. The method of claim 1, wherein the growth of the tumor is inhibited.

5. The method of claim 1, wherein the tumor volume is reduced.

6. The method of claim 1, wherein the molecule is delivered by intratumoral injection.

7. A method of detecting an IL13-specific receptor in a tissue specimen comprising normal cells or tumor cells, comprising the steps of:
(a) contacting a portion of the specimen with a labeled IL13 receptor-binding molecule under conditions suitable for binding of the IL13 receptor-binding molecule to an IL13 receptor for a period of time sufficient to allow said binding;
(b) washing the specimen sample portion of step a under conditions suitable for removing unbound IL13 receptor-binding molecule; and
(c) detecting the presence or absence of bound, labeled IL13 receptor-binding molecule to the specimen portion of step (b).

8. The method of claim 7, wherein the specimen portion of step a is preincubated in the presence or absence of IL4.

9. A method of imaging tumor cells having IL13-specific receptors in vivo in a mammalian subject comprising the steps of:
(a) delivering an imaging-effective amount of labeled IL13 receptor-binding molecule into the subject; and
(b) evaluating the distribution of the labeled IL13 receptor-binding molecule into the subject.

10. A pharmaceutical composition for inhibiting in vivo the growth of a tumor bearing an IL13-specific receptor comprising a molecule having an IL13 receptor-binding moiety and a cytotoxic moiety in a pharmaceutically acceptable carrier.

11. The pharmaceutical composition of claim 10, wherein the molecule is a chimeric molecule comprising human IL13 receptor-binding moiety and a cytotoxic moiety selected from the group consisting of PE3QQR, PE4E, and modified Diptheria toxin.

12. A kit for the in vivo or in vitro identification of cells bearing IL13-specific receptors comprising a compound comprising a portion of interleukin 13, the portion being capable of binding to an IL13-specific receptor to a greater extent than IL4 binds to the receptor.

13. An isolated polynucleotide fragment comprising a coding region for an IL13-specific receptor.
